(19) United States
(12) Patent Application Publication
(10) Pub. No.: US 2003/0097170 A1
(43) Pub. Date: May 22, 2003

(54) Implantation device for an aorta in an aortic arch

(75) Inventors: Holger Friedrich, Dresden (DE); Ajay Chavan, Hannover (DE); Axel Haverich, Isernhagen (DE)

Correspondence Address: WILLIAM COLLARD, COLLARD & ROE, P.C., 1077 NORTHERN BOULEVARD, ROSLYN, NY, 11576, US

(73) Assignee: CURATIVE AG

(21) Appl. No.: 10/252,777

(22) Filed: Sep. 23, 2002

(30) Foreign Application Priority Data
Sep. 25, 2001 (DE) 201 15 706.3
Aug. 6, 2002 (EP) 02017509.07

(51) Int. Cl. A61F 2/06

(52) U.S. Cl. 623/1.13; 623/1.16

(57) ABSTRACT

A stent made of a self-expandable or balloon-expandable metal mesh fabric that can be widened to a diameter larger than that of the descending aorta. The stent has a flexible lining of vessel-replacing material, wherein the lining covers it at least partly in longitudinal direction and forms a tubular segment which projects beyond the end of the stent at least at one end and which functions as the vascular prosthesis. Such implantable arrangements (1) make it possible to achieve short surgical times, especially during the treatment of aortic arch aneurysms involving the cephalic arteries.

### CROSS REFERENCE TO RELATED APPLICATIONS

### BACKGROUND

1. Field of the Invention

The invention relates to an arrangement for implantation in an aorta preferably in an aortic arch. The invention is used for treatment of aortic arch aneurysms and can be used during open-chest surgeries.

2. The Prior Art

From practice, several implantation arrangements are known for treatment of stenosis tissue damage. One of the arrangements, for example, is a stent disposed permanently in the vessel. This stent is a spreading element made of self-expandable or balloon-expandable metal mesh fabric. It has been found, however, that a vascular stent becomes coated after several weeks by a new vascular inner wall, which can lead to a restenosis in narrow vessels (residual stenosis).

U.S. Pat. No. 5,213,580 describes a balloon catheter with a coating of tissue-compatible plastic for treatment of vascular segments. This plastic seal is used to ensure that the vascular segment is held open to a constant extent after removal of the balloon catheter. One disadvantage is that it is difficult to fasten the plastic coating in the body vessel and furthermore, an area much larger than the treated tissue region must usually be sealed therewith.

German Patent 19731834 A1 describes an implantation device for treatment of damaged or diseased tissue in the region of the inner wall of hollow organs, especially for treatment of a dissection in body vessels. This implantation device has a self-expandable or balloon-expandable stent which is insertable into the vessel and fixed to the vessel wall with fastening means such as pins or clips. This device is used to enable strictly local treatment of damaged or diseased tissue of the inner wall of hollow organs while reducing the tissue stress to a minimum. The vascular prosthesis is made of a tissue-compatible material such as porous plastic or Nitinol. This arrangement has a disadvantage in that it has an extended surgery time wherein the duration of surgery becomes critical due to introduction of the fastening means, thus leading to an increased risk of brain injury, especially when it is used in the aortic arch.

German Utility Model 9116881 relates to a stent with a metal mesh scaffold. This metal mesh scaffold has a flexible jacket of tissue-compatible material. These stents are also known as stent grafts. Such a stent graft is unsuitable for treatment of aortic arch aneurysms involving the cephalic coronary vessels. A further disadvantage is that, when using a conventional stent graft or of vascular prosthesis to replace the vessel, it is not possible to circumvent preparation of the descending aorta and the associated lengthy surgical procedure.

### SUMMARY

One object of the invention is to provide for implantation into an aortic arch. This area of implantation permits simple and rapid simultaneous treatment of the descending aorta without additional time consumption or even a second surgical procedure. Another object is to shorten the time spent on anastomosis far from the heart. Yet another object is for the arrangement to be suitable as a therapeutic means for treatment of aortic arch aneurysms and for preventing an arch rupture.

This object is achieved by using a stent for implantation in an aorta, especially in the region of the aortic arch. This stent is made of a self-expandable or balloon-expandable metal mesh fabric that can be widened to a diameter larger than that of the descending aorta. This stent has a flexible lining of vessel-replacing material. This lining is attached to the inner and/or outer wall of the stent and covers it at least partly in longitudinal direction. The lining forms a tubular segment which projects beyond the end of the stent at least at one end and which functions as the vascular prosthesis

The device can be used to treat aortic arch aneurysms and to prevent subsequent ruptures. Since the arrangement requires neither an additional fastening means nor a prepared descending aorta, it can be introduced rapidly and in a relatively uncomplicated manner into the damaged vessel. The arrangement can be used for treatment of vessel bends, such as the aortic arch. The region of the cephalic arteries can be bridged with a single implant, namely the inventive arrangement, in the case of bilateral aneurysms. The free end of the tubing segment can be connected in standard manner to a further prosthesis or directly to the aortic tissue.

The lining of the stent and the tubing segment projecting beyond the stent can be made of a flexible tissue-compatible plastic.

The lining of the stent and the tubing segment projecting beyond the stent are made from different materials. This design is to achieve better adaptation of the implantable arrangement to the various requirements in the region of the aortic arch and within the descending aorta.

The lining of the stent should be made of Dacron and the projecting tubing segment should be made of material that has the same density as blood or is coated to achieve blood density.

To ensure that the stent with the lining does not block vascular outlets that may be located in this region, only between 20% to 90% of the stent should be covered with lining material in the longitudinal direction.

### BRIEF DESCRIPTION OF THE DRAWINGS

Other objects and features of the present invention will become apparent from the following detailed description considered in connection with the accompanying drawings which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1 shows a bilateral aneurysm of the aortic arch;

FIG. 2 shows a schematic diagram of an inventive implantable arrangement; and

FIG. 3 shows a schematic diagram of an inventive arrangement in situ in an aorta.

### DETAILED DESCRIPTION

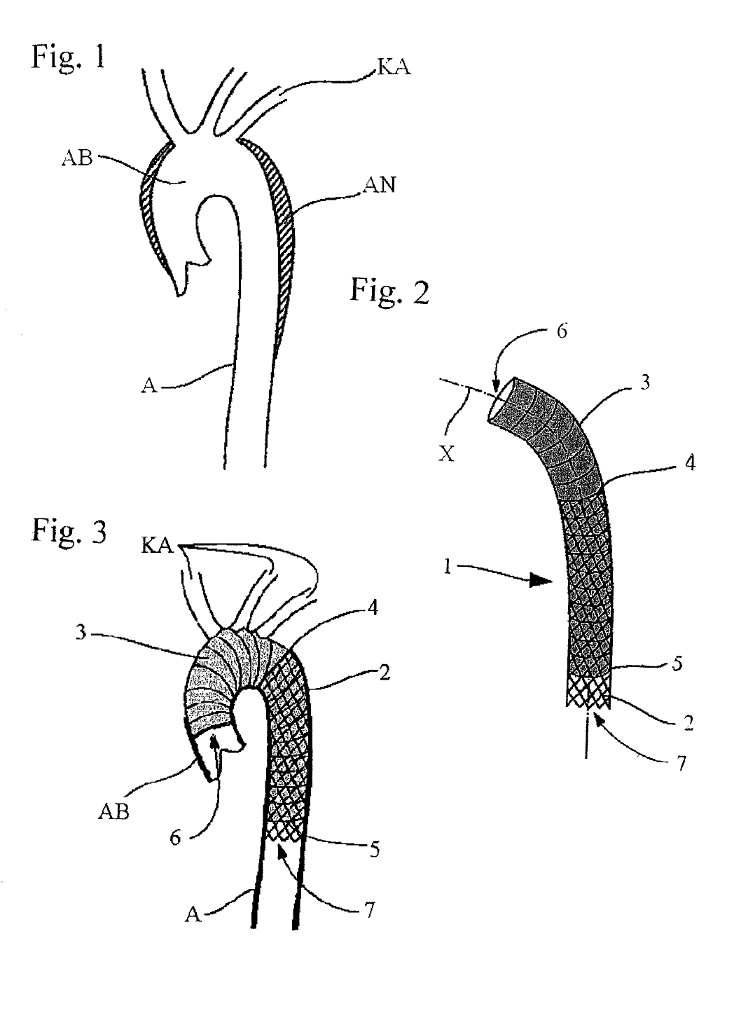

FIG. 1 illustrates an aorta in which aneurysms AN are present in the aortic arch AB on both sides of the cephalic arteries KA.

FIG. 2 shows an inventive implantable arrangement 1 which comprises a stent 2 of self-expandable tubular metal mesh fabric of Nitinol. This mesh is in the region between proximal end 4 and distal end 5 of a stent relative to a heart. This mesh is lined on its outside with Dacron having the same density as blood. Flexible tubing segment 3 functioning as a vascular prosthesis is formed by the extension of the lining of the same material disposed on a proximal part of stent 2. The longitudinal axis of implanted arrangement 1 is denoted by X. To prevent blockades of the vessels branching off from the aorta, the lining of stent 2 is expediently formed over only 80% of longitudinal direction X. The specific requirements for the length of stent 2 and of the lining as well as of free tubing segment 3 can be determined only after exact diagnosis of the damage in the region of the aortic arch.

For open-chest treatment of aortic arch aneurysm, an aortic arch AB is cut away, directly above the aortic valve and directly behind the cephalic arteries KA. The aortic tissue is then discarded. Using a suitable set of implantation instruments, the implantable arrangement with the distal end 7 formed by stent 2 is introduced into the thoracic portion of the descending aorta and released. This release results in stent 2, pressing against this descending artery to a diameter larger than the inner wall of the aorta by virtue of the memory property of the Nitinol. After the release of stent 2, which is covered partly with tissue of aorta A, it is implanted in the descending aorta, so that it ends with its distal end 7 disposed a few centimeters under the resection point. Stent 2 does not need any other fastening means at its distal end 7. At the top, free tubing segment 3 of implantable arrangement 1 is then available to the surgeon. Via this tubing segment 3, it is possible, as illustrated in FIG. 3, to bridge over corresponding bends and free spaces to replace aortic arch AB. Distal end 6 of tubing segment 3 can then be reconnected to the residual tissue of the aorta via a suture.

Furthermore, for complete treatment of the aortic arch aneurysm, a second, less complex vascular prosthesis can be introduced into the aorta at proximal end 6 of tubing segment 3. This device can then be fastened in a standard manner to proximal end 6 of tubing segment 3. Self-expandable stent 2 can be introduced into the descending aorta, which is firmly anchored to the tissue by virtue of overextension and friction. This design saves having to use a large suture and thus further surgical time. This time savings helps to avoid endangering the life of the patient. During this surgery, the patients must be artificially cooled to 17° C. To prevent brain damage, the surgical time should be limited to a maximum of 50 minutes this is because artificial circulation is impossible.

Accordingly, while at least one embodiment of the present invention has been shown and described, it is to be understood that many changes and modifications may be made thereunto without departing from the spirit and scope of the invention as defined in the appended claims.

What is claimed is:

1. An implantation device for an aorta in an aortic arch comprising:
a) a stent made from self-expandable metal mesh fabric that can be widened to a diameter larger than that of the aorta;
b) a flexible lining of vessel replacing material wherein said flexible lining is attached to an inner and an outer wall of said stent wherein said flexible lining contains a tubular segment which projects beyond an end of said stent and functions as a vascular prosthesis.

2. An implantation device for an aorta in an aortic arch comprising:
a) a stent made from balloon expandable metal mesh fabric that can be widened to a diameter larger than that of the aorta;
b) a flexible lining of vessel replacing material wherein said flexible lining is attached to an inner and an outer wall of said stent wherein said flexible lining contains a tubular segment which projects beyond an end of said stent and functions as a vascular prosthesis.

3. The device of claim 1, wherein said flexible lining is made from tissue compatible plastic.

4. The device of claim 2, wherein said flexible lining is made from tissue compatible plastic.

5. An arrangement as in claim 1, wherein said flexible lining of said stent and said tubing segment, projecting beyond said stent, are each made from different materials.

6. An arrangement as in claim 2, wherein said flexible lining of said stent and said tubing segment, projecting beyond said stent, are each made from different materials.

7. The device as in claim 5, wherein said lining is made from dracon and said protecting tubing segment is made of material having a density substantially similar to blood.

8. The device as in claim 6, wherein said lining is made from dracon and said protecting tubing segment is made of material having a density substantially similar to blood.

9. The device as in claim 1, wherein said lining covers between 20% and 90% of said stent, extending in a longitudinal direction from a distal end relative to a user's heart.

10. The device as in claim 2, wherein said lining covers between 20% and 90% of said stent, extending in a longitudinal direction from a distal end relative to a user's heart.
